(19) United States
(12) Patent Application Publication
(10) Pub. No.: US 2025/0161660 A1
(43) Pub. Date: May 22, 2025

(54) LINE DEVICE FOR CONDUCTING A BLOOD FLOW FOR A HEART SUPPORT SYSTEM, HEART SUPPORT SYSTEM, AND METHOD FOR PRODUCING A LINE DEVICE

(71) Applicant: KARDION GMBH, Stuttgart (DE)

(72) Inventors: Hardy Baumbach, Stuttgart (DE); Inga Schellenberg, Stuttgart (DE); David Minzenmay, Stuttgart (DE)

(21) Appl. No.: 19/030,520

(22) Filed: Jan. 17, 2025

Related U.S. Application Data
(63) Continuation of application No. 17/057,411, filed on Jun. 1, 2021, now Pat. No. 12,201,823.

(30) Foreign Application Priority Data
May 30, 2018 (DE) 102018208550.1

(51) Int. Cl. A61M 60/859; A61M 60/178; A61M 60/216; B21C 37/04

(52) U.S. Cl. CPC A61M 60/859; A61M 60/178; A61M 60/216; B21C 37/047; A61M 2205/0266; A61M 2207/00

(57) ABSTRACT

The invention relates to a line device (105) for conducting a blood flow for a heart support system. The heart support system has a head unit and an outlet unit. The line device (105) has a main part (205). The main part (205) has, at a first end, a first attachment section (210) for attaching the line device (105) to the head unit and, at a second end, a second attachment section (215) for attaching the line device (105) to the outlet unit. Furthermore, the main part (205) has a mesh section (220) between the attachment sections (210, 215), wherein the mesh section (220) has a mesh structure (230) formed from at least one mesh wire (225). In addition, the main part (205) has an inlet section (235), arranged in the first attachment section (210), for introducing the blood flow into the main part (205).

### INCORPORATION BY REFERENCE TO ANY PRIORITY APPLICATIONS

The present application is a continuation of U.S. application Ser. No. 17/057,411 filed on Jun. 1, 2021, which is a National State entry of PCT Patent Application No. PCT/EP2019/064138, filed on May 30, 2019, titled LINE DEVICE FOR CONDUCTING A BLOOD FLOW FOR A HEART SUPPORT SYSTEM, HEART SUPPORT SYSTEM, AND METHOD FOR PRODUCING A LINE DEVICE, which is an International Application of and claims the benefit of priority to German Patent Application No. 102018208550.1, filed on May 30, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND

### Field

The invention relates to a device or a method for conducting a blood flow for a heart support system.

### Description of the Related Art

Heart support systems, in particular left ventricular support systems, can be differentiated with regard to their position on the heart and their access to blood circulation. Long-term support systems can be positioned at the apex of the heart (transapically) and bridge the left heart chamber by pumping blood from the apex of the heart through a tube directly into the aorta. Another type of access can be used in particular for short-term support of the heart; for example, the heart support system can be a ventricular support system as a bridging measure for bridging until transplantation (bridge to decision, bridge to transplant). In this case, the natural aortic valve can be used to create a connection between the pump inlet and the pump outlet. In such an arrangement of the heart support system, the aorta can be used as an access route (transaortally) within the scope of minimally invasive surgery, and sternotomy can be avoided.

### SUMMARY

The invention is based on the object of specifying an improved line device for a heart support system, in particular with permanent connection reliability and suitable flexibility, as well as a method for its production.

In light of this background, the approach presented here presents a line device for conducting a blood flow for a heart support system, a heart support system, and a method for producing a line device according to the main claims. Advantageous developments and improvements of the device specified in the independent claim are possible by means of the measures listed in the dependent claims.

This approach presents a line device for conducting a blood flow for a heart support system, e.g., a left ventricular heart support system. The line device can be used as a flow channel in which the blood flow from the pump inlet in a left heart chamber can be conducted to the pump outlet within an aorta. The line device can have two attachment sections for attaching further components of the heart support system, and also a mesh section of at least one mesh wire arranged between the attachment sections. An advantageous ratio of flexibility and stiffness of the line device can be adjusted by means of the mesh structure of the mesh section. This is advantageous in order to allow transfemoral surgery (access via the groin) in order to implant the heart support system.

A line device for conducting a blood flow for a heart support system is presented. The heart support system has a head unit and an outlet unit. The line device has a main part, wherein the main part has, at a first end, a first attachment section for attaching the line device to the head unit and, at a second end, a second attachment section for attaching the line device to the outlet unit. The main part furthermore has a mesh section between the attachment sections. The mesh section has a mesh structure formed from at least one mesh wire. In addition, the main part has an inlet section, arranged in the first attachment section, for introducing the blood flow into the main part.

The line device can, for example, be formed from a biocompatible material and understood as a flow channel for the flexible connection of components of a heart support system for conducting a blood flow between the ventricle and the blood vessel. The line device can be used as a suction tube of the heart support system in order to introduce the blood flow and to conduct it further to an outlet section of the heart support system. For example, the heart support system can be understood to be a left ventricular support system (LVAD, left ventricular assist device) or another ventricular support system (VAD, ventricular assist device). The main part of the line device can, for example, be formed as a hollow cylinder and essentially have a pipe geometry. The main part can be formed in one piece or be constructed modularly; the main part can, for example, also be composed of the first attachment section, the mesh section, and the second attachment section. The first attachment section at a first end of the main part can be understood as a distal attachment section and be, for example, arranged in the implanted state of the heart support system as a left ventricular support system in the left heart chamber. The second attachment section at a second end of the main part can be understood as a proximal attachment section and be, for example, arranged in the implanted state of the left ventricular support system in the aorta. A head unit of the heart support system, e.g., a sensor assembly, can be attached to the first attachment section. The mesh section can, for example, be a main part section with a braided mesh structure for adjusting the flexibility and stiffness of the line device, wherein a stiffness of the mesh section is dependent on the mesh structure. Adjusting a predefined stiffness is advantageous in order to allow use of the line device during transfemoral surgery, for example. The flexibility and the stiffness of the mesh section can be adjusted, for example, by means of a number, a stiffness, or a material thickness of the at least one mesh wire, as well as by means of a mesh pattern of the mesh structure. The inlet section can be realized, for example, by a multi-part window in the first attachment section in order to allow the blood flow to be introduced into the main part of the line device.

According to one embodiment, at least one of the attachment sections can have at least one eyelet for threading an end of the at least one mesh wire in order to connect the mesh section to the respective attachment section. For example, the first attachment section can have the at least one eyelet, or the second attachment section can have the at least one eyelet, or the first attachment section and the second attachment section can respectively have at least one eyelet. The eyelet can, for example, be a hook eye or it can be realized as a bore in the respective attachment section. The eyelet can, for example, be realized on the side of the respective attachment section facing the mesh section. At least one of the attachment sections can also have a plurality of eyelets in order to, for example, connect several wire loops of the mesh wire to the respective attachment section. The mesh section can advantageously be easily and securely connected to the attachment section by means of the at least one eyelet.

In addition, according to one embodiment, the first attachment section can have at least one merlon. The merlon can in particular be arranged on the side of the first attachment section facing the mesh section. Additionally or alternatively, the second attachment section can have at least one merlon. In particular, the merlon can be arranged on the side of the second attachment section facing the mesh section. The at least one merlon can, for example, be formed as a projection of the respective attachment section. The merlon can, for example, also be formed to engage in the mesh section or on the mesh section. The at least one merlon can advantageously additionally fix the mesh section.

If at least one of the attachment sections according to one embodiment has at least one merlon, the at least one eyelet can be formed in the merlon. The eyelet can, for example, be designed to allow tangential threading of the mesh wire. The eyelet can, for example, have a longitudinal extension axis, which extends tangentially with respect to a circumference of the attachment section. At least one of the attachment sections can also have a plurality of merlons, wherein in this case at least one of the merlons or several merlons can have the at least one eyelet. The merlons can, for example, be arranged circumferentially and evenly spaced around the circumference of the attachment section.

The mesh structure of the mesh section can be formed as a diamond lattice according to one embodiment. The mesh structure can, for example, be braided from the at least one mesh wire, wherein the diamond lattice is formed from meshes of the mesh wire. The diamond shape can correspond to a standard shape of a vascular stent, which is advantageous with respect to the production of the mesh structure.

Furthermore, according to one embodiment, the mesh section can be formed to accommodate and/or guide a cable element of the heart support system. The cable element can, for example, be a section of a cable for signal and energy transmission. If the head unit of the heart support system has a sensor, for example, the cable element can be part of a sensor cable. The cable element can, for example, be braided into the mesh structure, or the cable element can, for example, be guided circumferentially around the mesh section along the mesh section; in this case, the cable element can be glued on, for example. This embodiment advantageously allows compact design. The mesh section can advantageously also provide mechanical protection from cable breakage.

If the mesh section is formed to accommodate the cable element, the mesh structure according to one embodiment can be formed from the at least one mesh wire and the cable element. The cable element can, for example, additionally be braided into the mesh structure. If the mesh structure is formed from at least two mesh wires, the cable element can also be used instead of one of the two mesh wires. For example, the mesh structure can in this case be formed, and one of the mesh wires can subsequently be removed and replaced by the cable element. This embodiment is also advantageous in terms of a compact design and protection of the cable element from cable breakage.

In addition, at least the mesh section can according to one embodiment be formed from a shape memory material. The shape memory material can be a biocompatible shape memory polymer, or a biocompatible shape memory alloy, such as Nitinol for example. Furthermore, the entire main part can also be manufactured from the shape memory material. The use of Nitinol as a shape memory material is advantageous since the Nitinol material is a proven material in medicine, in particular in the field of cardiovascular medicine, e.g., for heart valve prostheses, stents, and vascular prostheses, and, due to its biocompatibility and the shape memory property, allows even complex structures to be realized in a small installation space.

According to one embodiment, the mesh section extends over at least half of the main part in order to adjust the stiffness of the main part. This is advantageous with regard to implantation of the line device, in particular in the case of transfemoral access, in order to make possible a predefined ratio of flexibility and stiffness of the line device as a result of the forming the mesh section. The flexibility of the line device can be advantageous, for example, when pushing through an aortic bend, and the stiffness can advantageously prevent the line device from kinking when pushing through a blood vessel.

According to one embodiment, the line device can also have a sealing layer. The sealing layer can be arranged on or in the mesh section and be formed to fluid-tightly seal the mesh section. The fluid-tight sealing of the mesh section by means of the sealing layer is advantageous for conducting the blood flow in order to conduct the blood flow to the outlet unit without loss. The sealing section can be realized, for example, by casting or injection-molding the mesh section with a flexible plastic, such as polyurethane or silicone.

In addition, the mesh section can according to one embodiment have a bending point. The mesh section can in particular be bent at an obtuse angle at the bending point. The bending point can, for example, be formed in the center of the mesh section or be arranged closer to the first attachment section than to the second attachment section. The mesh section can, for example, have a first longitudinal axis between one end in the direction of the second attachment section and the bending point and have a second longitudinal axis obliquely to the first between the bending point and a second end in the direction of the first attachment section. At the bending point, an angle between the first and the second longitudinal axis can, for example, be between 20 degrees and 30 degrees, in particular 26 degrees. The bending point can, for example, be formed to give the main part a curved shape corresponding to human anatomy in order to allow the inlet section to be positioned in the center of a heart chamber in order to advantageously prevent the inlet unit from being sucked to a heart chamber wall.

Furthermore, according to one embodiment, an inner diameter of the main part can change from the first attachment section to the second attachment section. For example, a cross section of the inner diameter can taper in the direction of the second attachment section. The change in the inner diameter of the main part can advantageously improve the flow properties of the introduced blood flow.

According to one embodiment, the inlet section can have at least one inlet opening cut in the first attachment section. The inlet opening can, for example, be formed rectangularly or as a rectangle with a circular arc in the direction of the structural section. The inlet section can also have several inlet openings, e.g., three inlet openings. In this case, the inlet openings can be evenly spaced, for example, wherein, between two adjacent inlet openings, a narrow bridge can, for example, connect the first attachment section to the structural section. By forming at least one inlet opening, which can be cut in or into the main part, an additional structural element for introducing the blood flow can advantageously be dispensed with, which is advantageous with regard to a compact design.

A heart support system is also presented. The heart support system can have a head unit, an outlet unit, and an embodiment of the aforementioned line device. The line device can be arranged between the head unit and the outlet unit and connected to the head unit and the outlet unit.

A method for producing a line device for conducting a blood flow for a heart support system is also presented. The heart support system can have a head unit and an outlet unit. The method comprises the following steps:

forming a main part made of a semi-finished product made of a shape memory material, wherein the main part has, at a first end, a first attachment section for attaching the line device to the head unit and, at a second end, a second attachment section for attaching the line device to the outlet unit, wherein the main part has a mesh section between the attachment sections, wherein the mesh section has a mesh structure formed from at least one mesh wire, wherein the main part has an inlet section, arranged in the first attachment section, for introducing the blood flow into the main part; and

heat treating the formed main part in order to emboss a predefined shape into the main part.

By carrying out the aforementioned method, an embodiment of the aforementioned line device can advantageously be produced.

### BRIEF DESCRIPTION OF THE DRAWINGS

Exemplary embodiments of the approach presented here are shown in the drawings and explained in more detail in the following description. They show:

FIG. 1 a schematic illustration of a heart support system with a line device for conducting a blood flow according to an exemplary embodiment;

FIG. 2 a schematic illustration of a line device for conducting a blood flow for a heart support system according to an exemplary embodiment;

FIG. 3 a schematic illustration of a part of a line device according to an exemplary embodiment;

FIG. 4 a schematic illustration of an attachment section of a line device according to an exemplary embodiment;

FIG. 5 a schematic illustration of a part of an attachment section of a line device according to an exemplary embodiment;

FIG. 6 a schematic illustration of an attachment section of a line device according to an exemplary embodiment;

FIG. 7 a schematic illustration of a part of an attachment section of a line device according to an exemplary embodiment;

FIG. 8 a schematic illustration of a line device according to an exemplary embodiment;

FIG. 9 a schematic illustration of a line device according to an exemplary embodiment;

FIG. 10 a schematic illustration of a part of a line device according to an exemplary embodiment;

FIG. 11 a schematic illustration of a part of a line device according to an exemplary embodiment;

FIG. 12 a schematic illustration of a part of a line device according to an exemplary embodiment; and

FIG. 13 a flow diagram of a method for producing a line device for conducting a blood flow for a heart support system according to an exemplary embodiment.

### DETAILED DESCRIPTION

In the following description of favorable exemplary embodiments of the present invention, the same or similar reference signs are used for the elements which are shown in the various figures and have a similar effect, wherein a repeated description of these elements is omitted.

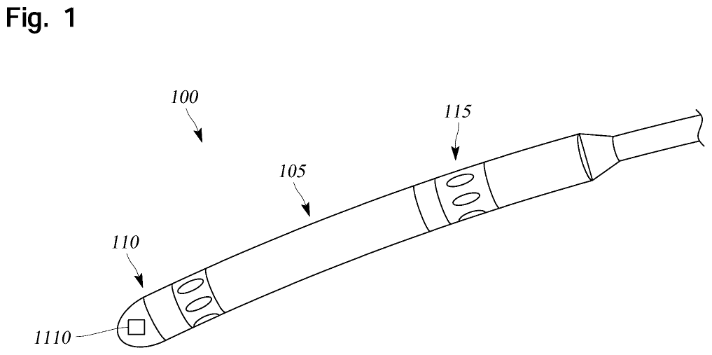

FIG. 1 shows a schematic illustration of a heart support system 100 with a line device 105 for conducting a blood flow according to an exemplary embodiment. Shown is a side view of the heart support system 100 as a whole system, which is designed here, by way of example, as a left ventricular support system 100. The heart support system 100 comprises a head unit 110, an outlet unit 115, and the line device 105. The line device 105 is arranged between the head unit 110 and the outlet unit 115 and is connected to the head unit 110 and the outlet unit 115. The line device 105 can also be referred to as a suction tube, which connects a pump inlet within a heart chamber to an outlet within the aorta in the implanted state of the heart support system 100.

The heart support system 100 has a cylindrical, elongated structure with a substantially constant outer diameter and rounded, tapered ends for easy positioning by means of a catheter in a blood vessel, e.g., the aorta.

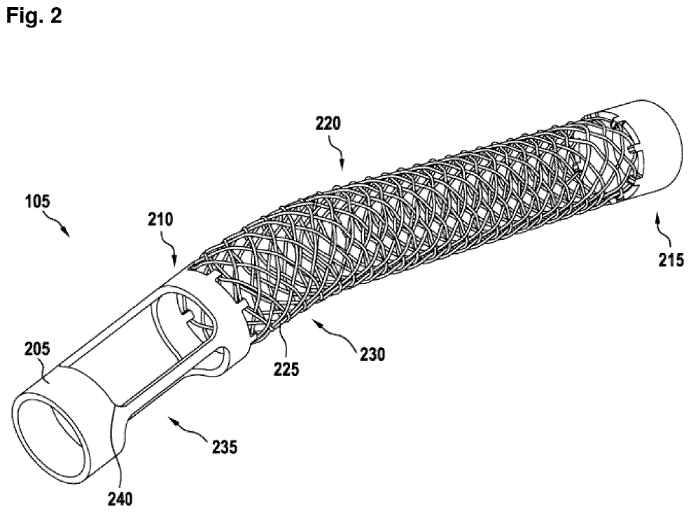

FIG. 2 shows a schematic illustration of a line device 105 for conducting a blood flow for a heart support system according to an exemplary embodiment in a side view. The line device 105 can also be referred to as a braided suction tube. The line device 105 has a main part 205. The main part 205 is formed in a tubular shape, for example. The main part 205 has, at a first end, a first attachment section 210 for attaching the line device 105 to a head unit of the heart support system and, at a second end, a second attachment section 215 for attaching the line device 105 to an outlet unit of the heart support system. In addition, the main part 105 has a mesh section 220 between the attachment sections 210, 215. The mesh section 220 has a mesh structure 230 formed from at least one mesh wire 225, wherein the main part 205 has an inlet section 235, arranged in the first attachment section 210, for introducing the blood flow into the main part 205. The line device 105 shown here can, for example, be similar or correspond to the line device shown in FIG. 1. The line device 105 is formed to be connectable to adjacent components of the heart support system as shown in FIG. 1. At the first attachment section 210, the line device 105 is formed to be connectable to a head unit of the heart support system and at the second attachment section 215, it is formed to be connectable to an outlet unit of the heart support system.

According to the exemplary embodiment shown here, the inlet section 235 has at least one inlet opening 240 cut in the first attachment section 210. Here, the inlet opening 240 is realized by way of example as a multi-part window. For the inflow of the blood, the inlet section 235 has three rectangularly formed inlet openings 240, which are rounded in the direction of the mesh section 220 in the form of a circular arc.

According to the exemplary embodiment shown here, the mesh structure 230 is formed as a diamond lattice. For this purpose, the at least one mesh wire 225 is braided as a lattice and has a plurality of diamond meshes that form the mesh structure 230.

The line device 105 is shown here with a braided flow channel as mesh section 220. According to one exemplary embodiment, at least the mesh section 220 is formed from a shape memory material. By way of example, the line device shown here is completely formed from Nitinol. By using Nitinol, the line device 105 is suitable not only for short-term use but also for a service life of over 10 years. In medicine, in particular in the field of cardiovascular medicine, Nitinol material is a proven material for heart valve prostheses, stents, and vascular prostheses, for example. Nitinol combines the advantages of biocompatibility and of the shape memory property that allows even complex structures to be realized in a small installation space as in the mesh section 220 shown here.

The mesh section 220 can be braided to the attachment points 210, 215. For this purpose, the attachment points 210, 215 have, for example, as shown here, a fastening element for threading a section of the mesh wire 225. Additionally or alternatively, the mesh section 220 can, for example, also be glued or soldered to the attachment points 210, 215.

According to the exemplary embodiment shown here, the mesh section 220 extends over at least half of the line device 105 in order to adjust the stiffness of the line device. The line device 105 is formed to allow transfemoral surgery (access via the groin). On the one hand, the line device 105 is thus formed flexibly enough to be able to be pushed through the aortic arch and has, on the other hand, a stiffness in order to be able to be pushed through the blood vessels in the axial direction without kinking. The requirements for flexibility and stiffness of the line device 105 in this regard are adjusted by means of the forming of the mesh section 220. The design of the braided structure adapts the ratio of flexibility and stiffness. Variables in this respect are the number of wire paths of the at least one mesh wire 225, a stiffness and a material thickness of the at least one mesh wire 225, as well as the mesh pattern of the mesh structure 230. The higher the number of wire paths of the at least one mesh wire 225 is, the stiffer is the mesh structure 230. The mesh wire 225 comprises, for example, 12 to 24 wire paths. The larger the wire diameter of the mesh wire 225 is, the stiffer is the mesh structure 230. For example, the wire diameter is between 0.1 millimeters and 0.3 millimeters. In addition, material properties of the mesh wire 225 are important: The higher the modulus of elasticity of the mesh wire 225 is, the stiffer is the mesh structure 230. The mesh wire 225 has an elasticity between 74 GPa and 83 GPa, for example. The mesh type of the mesh structure 230 is also important: the closer the meshes are in the mesh, the stiffer is the mesh.

In the exemplary embodiment shown here, the line device 105 is bent in the direction of the first attachment section 210, wherein the bend is by way of example formed as an obtuse angle with respect to a longitudinal axis of the line device 105. The bending can be realized by heat treating the mesh section 220 made of Nitinol. Due to the shape memory properties of the Nitinol, the line device 105 can be formed by a waveform of the mesh section 220 corresponding to human anatomy in order to allow positioning of the inlet opening of the inlet section 235 in the first attachment section 210 in the middle of the heart chamber.

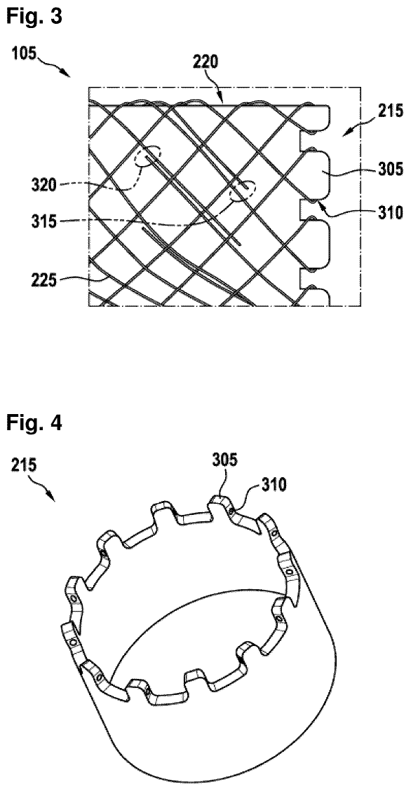

FIG. 3 shows a schematic illustration of a part of a line device 105 according to an exemplary embodiment. A section of the mesh section 220 connected to the second attachment section 215 is shown in a side view. The first attachment section can be formed like the second attachment section 215 shown here by way of example. The part of the line device 105 shown here is a detail of a similar or corresponding line device as described with reference to FIG. 2.

According to this exemplary embodiment, at least one of the attachment sections, in this case by way of example the second attachment section 215, has at least one eyelet 310 for threading an end of the at least one mesh wire 225 in order to connect the mesh section 220 to the corresponding attachment section. In addition, the first attachment section and/or the second attachment section 215 has at least one merlon 305 as an attachment point for the mesh section 220. The merlon 305 is arranged in particular on the side of the attachment section 215 facing the mesh section 220, as shown here. Optionally, the at least one eyelet 310 is formed in the at least one merlon 305, as shown here.

By way of example, the second attachment section 215 has here a plurality of merlons 305. Each of the merlons 305 has an eyelet 310, through which a section of a mesh wire 225 is guided. In the exemplary embodiment shown here, the structure of the mesh section 220 is formed by way of example from several mesh wires 225. Wire ends of the mesh wires are, for example, integrated into the mesh structure, as indicated by the markings 315 and 320. By means of the mesh wires 225, pump components adjacent through the first and the second attachment section 215, i.e., further components of the heart support system, such as the head unit and the outlet unit, can be connected to the line section 105 permanently and securely. For this purpose, an eyelet 310 into which the mesh wire 225 is threaded is provided for each wire loop of the at least one mesh wire 225. The eyelet 310 can be realized as a bore.

FIG. 4 shows a schematic illustration of an attachment section of a line device according to an exemplary embodiment. A second attachment section 215 is shown by way of example in a top view. The second attachment section 215 shown here corresponds or is similar to the attachment sections shown in the preceding figures. The at least one mesh wire can be braided into the second attachment section 215. As attachment points for the braiding-in of the mesh wire 225, the second attachment section 215 has a plurality of merlons 305 distributed rotationally evenly on the diameter of the second attachment section 215, i.e., spaced circumferentially evenly. The second attachment section 215 comprises, for example, 6 to 12 merlons 305; 12 merlons 305 are shown here. The eyelets 310 formed in the merlons 305 are realized as bores in the circumferential direction of the cylindrical second attachment section 215 in order to allow tangential threading of the mesh wire 225 in order to not increase the total diameter of the line device 105.

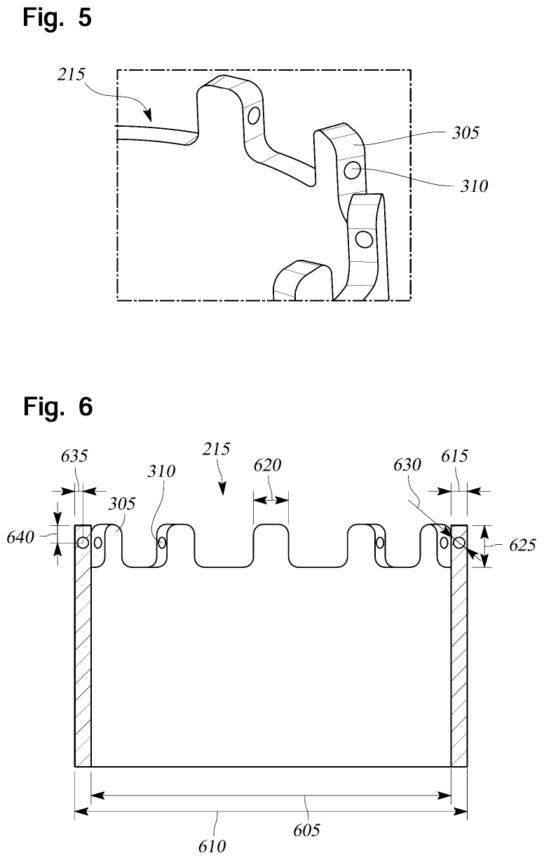

FIG. 5 shows a schematic illustration of a part of an attachment section 215 of a line device according to an exemplary embodiment. The part shown here is a detail of the second attachment section 215 shown in the preceding FIG. 4. In the detail shown here, each of the merlons 305 has an exposed eyelet 310 as a single attachment point, into which the mesh wire can be tangentially inserted. The eyelets 310 each run transversely with respect to the longitudinal axis of the line device through the merlons 305.

FIG. 6 shows a schematic illustration of an attachment section 215 of a line device according to an exemplary embodiment. The cross section of a second attachment section 215 is shown in order to illustrate exemplary dimensions of the second attachment section 215. The second attachment section 215 shown corresponds to or is similar to the second attachment section 215 of one of the figures described above. The first attachment section can be formed similarly or correspondingly. The second attachment section 215 has, for example, an inner diameter of 5.5 millimeters indicated by the marking 605 and an outer diameter of 6 millimeters indicated by the marking 610. The merlons 305 have a wall thickness of 0.25 millimeters, which the marking 615 indicates, a width of 0.5 millimeters indicated by the marking 620, and a height of 0.6 millimeters indicated by the marking 625. Each merlon 305 has an eyelet 310 with an inner diameter of 0.15 millimeters, as indicated by the marking 630. The distance from the outer wall of the merlon 305 to the center point of the eyelet 310 is, for example, 0.25 millimeters, as the marking 635 indicates, and the distance from the center point of the eyelet 310 to an upper free end of the merlon 305 is also 0.25 millimeters, as indicated by the marking 340. The bore diameter of each eyelet 310 is somewhat larger than the thickness of the mesh wire in order to allow a wire end to be inserted and the mesh wire to be braided in. The eyelets 310 can be drilled by means of a laser drilling method.

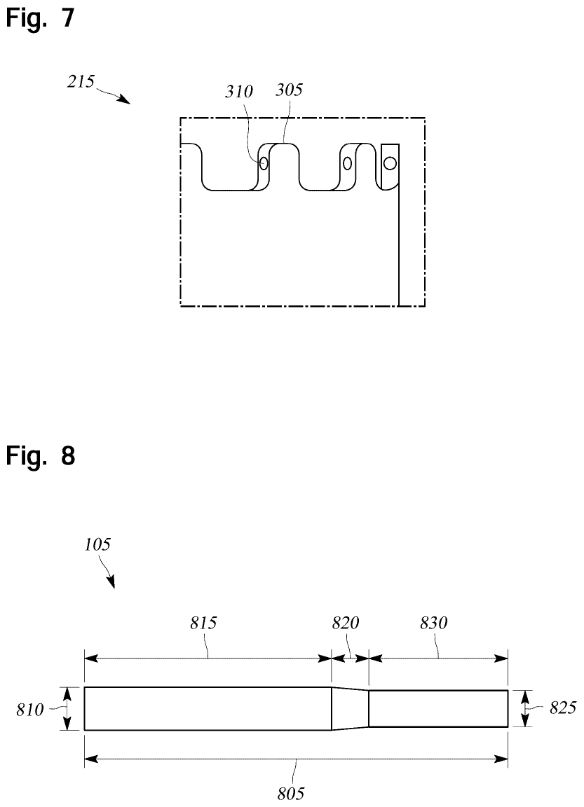

FIG. 7 shows a schematic illustration of a part of an attachment section of a line device according to an exemplary embodiment. The part shown here is a further detail of the second attachment section 215 shown in FIG. 4, wherein a side view of the detail is shown here. The exemplary shape of the eyelets 310 shown here and the arrangement of the eyelets 310 and merlons 305 allow radial tangential access. The eyelets 310 and the merlons 305 are arranged and dimensioned such that a radial-tangential laser access path is possible when producing the second attachment section 215.

FIG. 8 shows a schematic illustration of a line device 105 according to an exemplary embodiment. The line device 105 corresponds or is similar to the line device of one of the figures described above. Shown is a change in an inner diameter of the main part of the line device 105, which is illustrated using exemplary dimensions of the line device 105. The main part of the line device 105 has a length of 62 millimeters illustrated by the marking 805. According to the exemplary embodiment shown here, an inner diameter of the main part changes from the first attachment section to the second attachment section. The cross section of the first attachment section has a diameter of 6 millimeters, as indicated by the marking 810. The marking 815 indicates a length of 35 millimeters of a section of the main part with the first attachment section with an inner diameter of 6 millimeters throughout. In the section marked by the marking 820, which has a length of 5 millimeters and adjoins the section of the marking 815, the inner diameter of the line device 105 tapers from 6 millimeters to 5.5 millimeters, as indicated by the marking 825. Over the remaining length, indicated by the marking 830, of 22 millimeters of the main part of the line device 105 shown here, the inner diameter remains constant at 5.49 millimeters. The change in the inner diameter of the main part shown here can improve the flow properties of the blood flow. For this purpose, different diameters in the axial direction can be embossed into the line device 105 as shown here. The inner diameter can in particular be larger in the region with the first attachment section indicated by way of example by the marking 815 than in the region with the second attachment section indicated by the marking 830. In the region corresponding to the marking 815, a larger installation space can, for example, be available than in the region indicated by the marking 830 if the region 830 is, for example, enclosed by a further structural element, for example a sleeve, for inserting the heart support system into a catheter when implanting the heart support system. The braided contour of the mesh section of the line device 105 as a Nitinol element can be formed within the scope of a heat treatment into a predefined shape with, for example, different diameters as shown here. The embossing process in this case describes a plastic deformation without the occurrence of material failure.

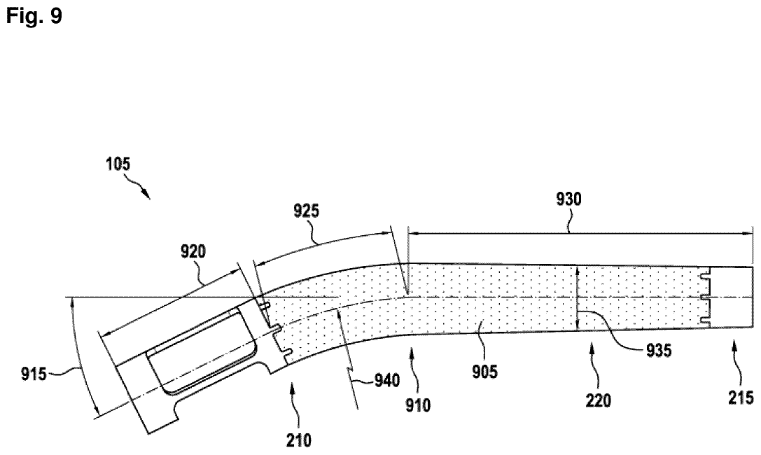

FIG. 9 shows a schematic illustration of a line device 105 according to an exemplary embodiment. A side view of the line device 105 is shown. By means of markings, this figure illustrates exemplary dimensions of the line device 105 as a suction tube. The line device 105 substantially corresponds to the line device described with reference to FIG. 2, with the exception of a sealing layer 905 arranged on the mesh section 220. According to the exemplary embodiment shown here, the line device 105 comprises the sealing layer 905. The sealing layer 905 is arranged on or in the mesh section 220. The sealing layer 905 is formed to seal the mesh section 220 in a fluid-tight manner. By way of example, the sealing layer 905 is shown here as a silicone casting.

According to the exemplary embodiment shown here, the mesh section 220 furthermore has a bending point 910. The mesh section 220 is in particular bent at an obtuse angle at the bending point, here by way of example by 26 degrees as indicated by the marking 915. The first attachment section 210 has a length of 15.4 millimeters indicated by the marking 920. The section, indicated by the marking 925, of the mesh section 220 with the bending point 910 has a length of 13.6 millimeters, and the remaining section of the main part with a second part of the mesh section 220 and the second attachment section has a length of 33 millimeters as indicated by the marking 930. At the point indicated by the marking 935, the mesh section has a bend of 2 degrees.

FIG. 10 shows a schematic illustration of a part of a line device according to an exemplary embodiment. A top view of a detail of the mesh section 220 with a sealing layer 905 is shown. The mesh section 220 is shown as a wire mesh structure, and is, by way of example, cast with a plastic as a sealing layer 905. The sealing layer 905 seals the mesh section 220 in a fluid-tight manner so that the blood flow in the inlet section can be sucked in and pumped through the line device 105, along the mesh section 220, into the outlet unit and thus into the aorta without loss. The sealing layer 905 is formed from a plastic, e.g., polyurethane or silicone, which is still soft enough after curing to withstand the movements of the line device 105 during operation of the heart support system. When the heart support system, in particular the line device 105, is pushed through the aortic arch during surgery, cracks are prevented from forming in the plastic of the sealing layer 905 as a result of a correspondingly selected material of the sealing layer 905.

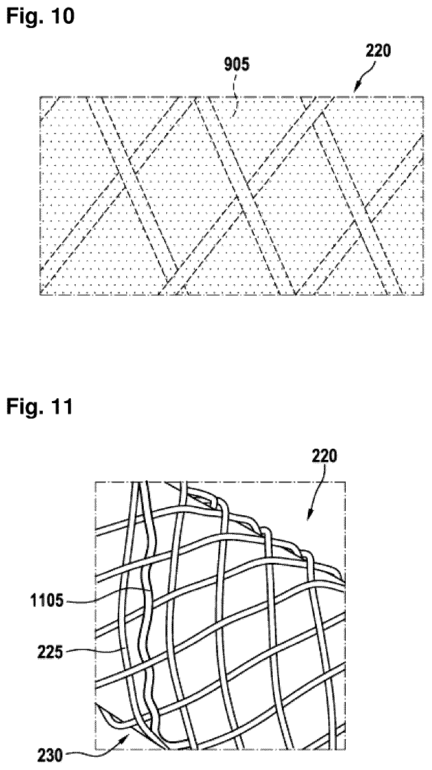

FIG. 11 shows a schematic illustration of a part of a line device according to an exemplary embodiment. A detail of a section of a mesh section 220 is shown. The mesh section 220 substantially corresponds to the mesh section 220 shown in the preceding figures. In addition, the mesh section 220 according to the exemplary embodiment shown here is formed to receive a cable element 1105 of the heart support system, and, additionally or alternatively, the mesh section 220 is formed to guide a cable element 1105 of the heart support system. As shown here, the mesh structure 230 is optionally formed from the at least one mesh wire 225 and the cable element 1105. The wire mesh of the mesh structure 230 thus comprises an additionally braided-in cable as a cable element 1105. The cable element 1105 can be designed as a section of a cable that allows an electrical data and energy connection from the head unit, e.g., a sensor tip, to the pump of the heart support system. If the head unit 110 of the heart support system has a sensor 1110, for example, the cable element 1105 can be part of a sensor cable. If the mesh structure 230 is formed from several mesh wires 225, a mesh wire 225 is optionally exchanged for the cable element 1105 and the cable element 1105 is braided in in a break-proof manner. If the mesh section 220 has a sealing layer, the sealing layer is an additional mechanical protection of the cable element 1105.

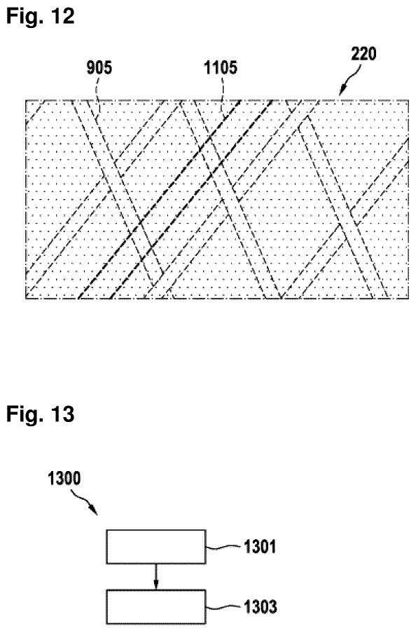

Alternatively, as a replacement of a Nitinol wire as a mesh wire 225, the cable element 1105 is already integrated in the weaving process during the production of the mesh structure 230. Used for this purpose is in particular a round cable, which has similar geometric diameters and mechanical properties as the mesh wire 225 and a thermal resistance for the subsequent heat treatment. Alternatively, the cable is furthermore guided along the outer or inner side of the mesh section without being braided in, as shown in FIG. 12.

FIG. 12 shows a schematic illustration of a part of a line device according to an exemplary embodiment. The illustration is similar or corresponds to the illustration of the detail of the line device described with reference to FIG. 10. In addition, the detail of the mesh section 220 shown here with the sealing layer 905 comprises the cable element 1105. The mesh section 220 is shown by way of example as a wire mesh with an applied and cast flat ribbon cable as a cable element 1105. The cable element 1105 is arranged circumferentially in the shape of a spiral around the longitudinal axis of the mesh section 220, it is glued here and cast or injection-molded with the sealing layer 905, i.e., surrounded by the sealing layer 905. In order to keep the mechanical stresses on the cable element 1105 low, the braided wire slope of the mesh wire is used here by way of example for routing the cable element 1105 in a spiral shape and for fastening the cable element 1105.

FIG. 13 shows a flow diagram of a method 1300 for producing a line device for conducting a blood flow for a heart support system according to an exemplary embodiment. The heart support system has a head unit and an outlet unit. The method 1300 comprises a step 1301 of forming and a step 1303 of heat treating. In step 1301 of forming, a main part is formed from a semi-finished product made of a shape memory material. The main part has, at a first end, a first attachment section for attaching the line device to the head unit and, at a second end, a second attachment section for attaching the line device to the outlet unit. The main part additionally has a mesh section between the attachment sections. The mesh section has a mesh structure formed from at least one mesh wire. Furthermore, the main part has an inlet section, arranged in the first attachment section, for introducing the blood flow into the main part. In step 1303 of heat treating, the main part formed in step 1301 is heat treated in order to emboss a predefined shape into the main part.

If an exemplary embodiment includes an “and/or” conjunction between a first feature and a second feature, this should be read to mean that the exemplary embodiment according to one embodiment comprises both the first feature and the second feature and according to another embodiment comprises either only the first feature or only the second feature.

What is claimed is:

1. A heart support system comprising:
a head unit;
an outlet unit;
a line device arranged between the head unit and the outlet unit and configured to conduct a blood flow for the heart support system, wherein the line device comprises:
a first end configured to attach to the head unit;
an inlet proximate to the first end and configured to introduce the blood flow into the line device;
a second end configured to attach to the outlet unit; and
a mesh section between the first end and the second end, wherein the mesh section comprises a mesh structure formed from at least one mesh wire,
wherein the mesh section is configured to receive and/or guide a cable element of the heart support system; and
a sensor connected to the cable element.

2. The heart support system according to claim 1, wherein the line device further comprises at least one eyelet configured to thread onto an end of the at least one mesh wire to connect the mesh section to at least one of the first end and the second end.

3. The heart support system according to claim 1, wherein the mesh structure is formed as a diamond lattice.

4. The heart support system according to claim 1, wherein the mesh structure is formed from the at least one mesh wire and the cable element.

5. The heart support system according to claim 1, wherein at least the mesh section is formed of a shape memory material.

6. The heart support system according to claim 1, wherein the mesh section extends over at least half of the line device to adjust a stiffness of the line device.

7. The heart support system according to claim 1, wherein the line device further comprises a sealing layer arranged on or in the mesh section, wherein the sealing layer is configured to seal the mesh section in a fluid-tight manner.

8. The heart support system according to claim 1, wherein an inner diameter of the line device changes between the first end and the second end.

9. The heart support system according to claim 1, wherein the inlet comprises at least one inlet opening.

10. A heart support system comprising:
a head unit;
an outlet unit;
a line device arranged between the head unit and the outlet unit and configured to conduct a blood flow for the heart support system, wherein the line device comprises:
a first end configured to attach to the head unit;
an inlet proximate to the first end and configured to introduce the blood flow into the line device;
a second end configured to attach to the outlet unit; and
a mesh section between the first end and the second end, wherein the mesh section extends over at least half of the line device to adjust a stiffness of the line device;
a cable element integrated into the mesh section; and
a sensor connected to the cable element.

11. The heart support system according to claim 10, wherein the line device further comprises a sealing layer arranged on or in the mesh section, wherein the sealing layer is configured to seal the mesh section in a fluid-tight manner.

12. The heart support system according to claim 10, wherein at least the mesh section is formed of a shape memory material.

13. The heart support system according to claim 10, wherein the mesh section is formed as a diamond lattice.

14. A heart support system comprising:
a head unit;
a sensor;
an outlet unit; and
a line device arranged between the head unit and the outlet unit and configured to conduct a blood flow for the heart support system, wherein the line device comprises:
a first end configured to attach to the head unit;
an inlet proximate to the first end, the inlet comprising at least one inlet opening;
a second end configured to attach to the outlet unit;
a mesh section between the first end and the second end;
a cable element arranged to spiral around a longitudinal axis of the mesh section and attached to the sensor.

15. The heart support system according to claim 14, wherein at least one of the first end and the second end comprises at least one eyelet configured to thread an end of the mesh section to connect the mesh section to at least one of the first end and the second end.

16. The heart support system according to claim 14, wherein the mesh section is formed from at least one mesh wire and the cable element.

17. The heart support system according to claim 14, wherein the line device further comprises a sealing layer arranged on or in the mesh section, wherein the sealing layer is configured to seal the cable element to the mesh section.

18. The heart support system according to claim 14, wherein an inner diameter of the line device changes between the first end and the second end.

19. The heart support system according to claim 14, wherein the at least one inlet opening is rectangular.

20. The heart support system according to claim 19, wherein the at least one inlet opening is evenly spaced around a circumference of the inlet.
